(19) United States
(12) Patent Application Publication
(10) Pub. No.: US 2019/0328365 A1
(43) Pub. Date: Oct. 31, 2019

(54) ULTRASOUND DIAGNOSIS APPARATUS AND STORAGE MEDIUM

(71) Applicant: CANON MEDICAL SYSTEMS CORPORATION, Otawara-shi (JP)

(72) Inventors: Koji KUROIWA, Kawasaki (JP); Eiji Goto, Utsunomiya (JP); Yoshihiro Oomori, Otawara (JP); Tomokazu FUJII, Nasushiobara (JP)

(73) Assignee: CANON MEDICAL SYSTEMS CORPORATION, Otawara-shi (JP)

(21) Appl. No.: 16/394,573

(22) Filed: Apr. 25, 2019

(30) Foreign Application Priority Data
Apr. 25, 2018 (JP) 2018-084474
Apr. 23, 2019 (JP) 2019-081866

(51) Int. Cl. A61B 8/08; A61B 8/00

(52) U.S. Cl. CPC A61B 8/5207; A61B 8/463; A61B 8/488; A61B 8/14; A61B 8/54; A61B 8/4488; A61B 8/5223; A61B 8/4444

(57) ABSTRACT

An ultrasound diagnosis apparatus according to an embodiment includes processing circuitry configured: to obtain information about a degree of deterioration of each ultrasound probe; and to display pieces of identification information of a plurality of ultrasound probes in a ranking order according to the information.

### CROSS-REFERENCE TO RELATED APPLICATIONS

This application is based upon and claims the benefit of priority from Japanese Patent Application No. 2018-84474, filed on Apr. 25, 2018 and Japanese Patent Application No. 2019-81866, filed on Apr. 23, 2019; the entire contents of which are incorporated herein by reference.

### FIELD

Embodiments described herein relate generally to an ultrasound diagnosis apparatus and a storage medium.

### BACKGROUND

Ultrasound diagnosis apparatuses are apparatuses configured to acquire ultrasound image data by transmitting and receiving ultrasound waves by using an ultrasound probe. There are different types of ultrasound probes, and an appropriate type of ultrasound probe is used for each medical examination. For example, a hospital or the like having an ultrasound diagnosis apparatus is equipped with a plurality of types of ultrasound probes having mutually-different shapes and mutually-different frequencies of ultrasound waves to be transmitted. In that situation, a user (e.g., a medical doctor or a medical technologist) of the ultrasound diagnosis apparatus uses an ultrasound probe selected in accordance with the purpose. Incidentally, ultrasound probes gradually become deteriorated during the use and, in some situations, image quality of the acquired ultrasound image data may be degraded.

### BRIEF DESCRIPTION OF THE DRAWINGS

FIG. 1 is a block diagram illustrating an exemplary configuration of an ultrasound diagnosis system according to a first embodiment;

FIG. 2 is a block diagram illustrating an example of an ultrasound diagnosis apparatus according to the first embodiment;

FIG. 3 is a drawing for explaining examples of a ranking process and a display process according to the first embodiment;

FIG. 4 is a drawing illustrating an example of displaying a ranking order according to the first embodiment;

FIG. 5 is a drawing illustrating another example of the displaying of the ranking order according to the first embodiment;

FIG. 6 is a flowchart for explaining a flow of a series of processes performed by the ultrasound diagnosis apparatus according to the first embodiment;

FIG. 7 is a drawing illustrating an example of displaying a ranking order according to a second embodiment;

FIG. 8 is a flowchart for explaining a flow of a series of processes performed by an ultrasound diagnosis apparatus according to the second embodiment;

FIG. 9A is a drawing illustrating an example of displaying a ranking order according to a third embodiment; and

FIG. 9B is a drawing illustrating another example of the displaying of the ranking order according to the third embodiment.

### DETAILED DESCRIPTION

An ultrasound diagnosis apparatus comprises processing circuitry. The processing circuitry is configured to obtain information about a degree of deterioration of each ultrasound probe. And the processing circuitry is configured to display pieces of identification information of a plurality of ultrasound probes in a ranking order according to the information.

Exemplary embodiments of the ultrasound diagnosis apparatus and a storage medium will be explained in detail below, with reference to the accompanying drawings.

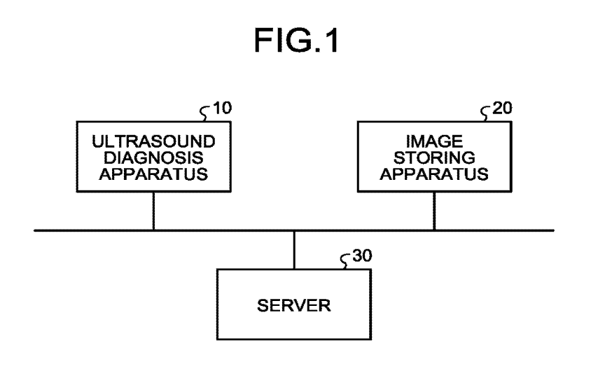

In a first embodiment, an ultrasound diagnosis system 1 including an ultrasound diagnosis apparatus 10 will be explained as an example. As illustrated in FIG. 1, the ultrasound diagnosis system 1 includes the ultrasound diagnosis apparatus 10, an image storing apparatus 20, and a server 30. Further, the ultrasound diagnosis apparatus 10, the image storing apparatus 20, and the server 30 are connected to one another via a network. FIG. 1 is a block diagram illustrating an exemplary configuration of the ultrasound diagnosis system according to the first embodiment.

The ultrasound diagnosis apparatus 10 is an apparatus configured to acquire ultrasound image data by transmitting and receiving ultrasound waves to and from an examined subject (hereinafter “patient”) P, by using an ultrasound probe. The ultrasound diagnosis system 1 may include two or more ultrasound diagnosis apparatuses 10. A configuration of the ultrasound diagnosis apparatus 10 will be explained later.

The image storing apparatus 20 is configured to store therein the ultrasound image data acquired by the ultrasound diagnosis apparatus 10. For example, the image storing apparatus 20 obtains the ultrasound image data from the ultrasound diagnosis apparatus 10 via the network and stores the obtained ultrasound image data into a memory provided either inside or outside of the apparatus. The server 30 is a server apparatus configured to manage a Hospital Information System (HIS), for example.

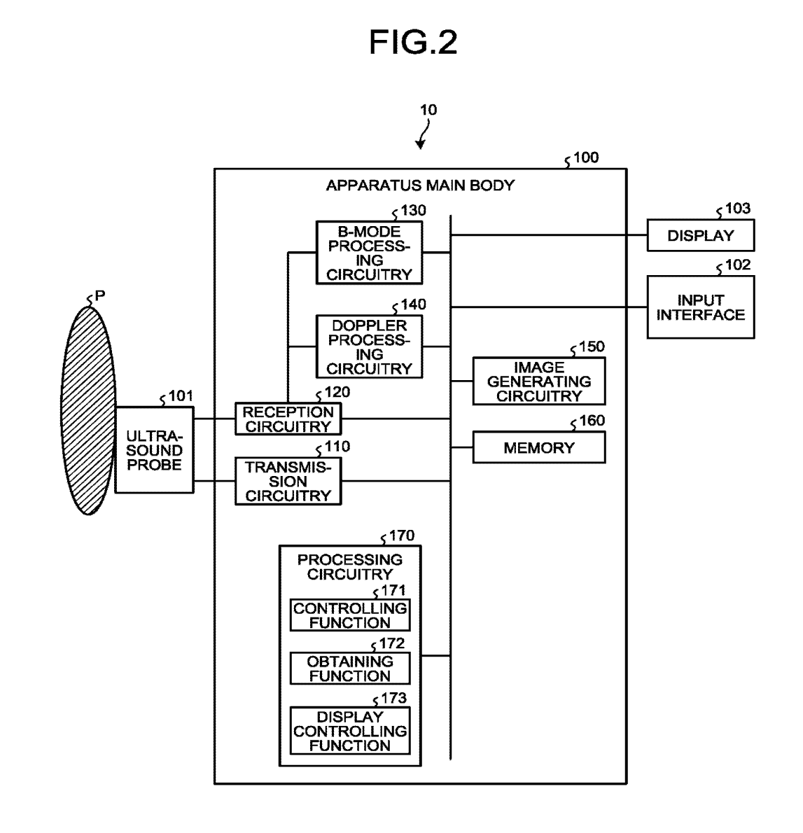

Next, a configuration of the ultrasound diagnosis apparatus 10 will be explained, with reference to FIG. 2. As illustrated in FIG. 2, the ultrasound diagnosis apparatus 10 according to the first embodiment includes an apparatus main body 100, an ultrasound probe 101, an input interface 102, and a display 103. Further, as illustrated in FIG. 2, the ultrasound probe 101, the input interface 102, and the display 103 are each connected to the apparatus main body 100.

FIG. 2 is a block diagram illustrating the exemplary configuration of the ultrasound diagnosis apparatus 10 according to the first embodiment.

The ultrasound probe 101 includes a plurality of transducer elements (piezoelectric transducer elements). The ultrasound probe 101 is configured to transmit and receive the ultrasound waves (to perform an ultrasound scan), while being in contact with the body surface of the patient P. The plurality of transducer elements are configured to generate the ultrasound waves on the basis of a drive signal supplied thereto from transmission circuitry 110 (explained later). The generated ultrasound waves are reflected on a plane of unmatched acoustic impedance in the body of the patient P and are received by the plurality of transducer elements as reflected-wave signals (reception echo) including a component scattered by a scattering member in a tissue, and the like. The ultrasound probe 101 is configured to forward the reflected-wave signals received by the plurality of transducer elements to reception circuitry 120.

In this situation, the ultrasound probe 101 connected to the apparatus main body 100 may be changed as appropriate. For example, the user (e.g., a medical doctor or a medical technologist) selects one of a plurality of ultrasound probes 101 that can be connected to the apparatus main body 100 in accordance with an examination target region of the patient P and connects the selected ultrasound probe 101 to the apparatus main body 100.

The ultrasound probe 101 connected to the apparatus main body 100 may be a two-dimensional ultrasound probe (a 2D array probe) including the plurality of transducer elements arranged in a matrix formation (a grid formation) or may be a one-dimensional ultrasound probe (a 1D array probe) including the plurality of transducer elements that are one-dimensionally arranged in a predetermined direction. Further, more than one ultrasound probe 101 may be connected to the apparatus main body 100.

For example, a hospital or the like having the ultrasound diagnosis apparatus 10 is equipped with a plurality of types of ultrasound probes 101 having mutually-different shapes and mutually-different frequencies of ultrasound waves to be transmitted. In one example, the hospital or the like is equipped with various types of ultrasound probes 101 such as a linear probe, a convex probe, a sector probe, an intracavity probe, and the like. In this situation, when performing a medical examination on a blood vessel near the body surface or the thyroid gland, the user performs an ultrasound scan by connecting the linear probe to the apparatus main body 100, for instance. As another example, when performing a medical examination on the abdomen, the user performs an ultrasound scan by connecting a convex probe to the apparatus main body 100. As yet another example, when performing a medical examination on the heart, the user performs an ultrasound scan by connecting a sector probe to the apparatus main body 100. As yet another example, when performing a medical examination on the heart via the esophagus, the user performs an ultrasound scan by connecting an intracavity probe such as a transesophageal echocardiography (TEE) probe or the like, to the apparatus main body 100.

The input interface 102 is configured to receive various types of input operations from the user, to convert the received input operations into electrical signals, and to output the electrical signals to processing circuitry 170. For example, after the ultrasound diagnosis apparatus 10 is started up, the input interface 102 receives an operation performed by the user to log in. Further, for example, the input interface 102 receives an operation to input patient information (a patient's ID, the gender of the patient, the age of the patient, examination results from the past, observations of medical doctors, and the like) about the patient subject to a medical examination. For example, the input interface 102 is realized by using a mouse, a keyboard, a trackball, a switch, a button, a joystick, a touchpad used for performing an input operation by touching an operation surface thereof, a touch screen in which a display screen and a touchpad are integrally formed, a contactless input circuit using an optical sensor, an audio input circuit, and/or the like. The input interface 102 does not necessarily have to include one or more physical operation component parts such as a mouse and a keyboard. For example, possible examples of the input interface 102 include an electrical signal processing circuit configured to receive an electrical signal corresponding to an input operation from an external input device provided separately from the apparatus main body 100 and to output the electrical signal to the processing circuitry 170.

The display 103 is configured to display a Graphical User Interface (GUI) used by the user of the ultrasound diagnosis apparatus 10 to input various types of setting requests through the input interface 102 and to display the ultrasound image data generated by the apparatus main body 100 and the like. For example, the display 103 is structured by using a liquid crystal display, a Cathode Ray Tube (CRT) display, or the like. The display 103 may be of a desktop type or may be configured with a tablet terminal or the like capable of wirelessly communicating with the apparatus main body 100.

The apparatus main body 100 is configured to generate the ultrasound image data on the basis of the reflected-wave signals received by the ultrasound probe 101. For example, as illustrated in FIG. 2, the apparatus main body 100 includes the transmission circuitry 110, the reception circuitry 120, B-mode processing circuitry 130, Doppler processing circuitry 140, image generating circuitry 150, a memory 160, and the processing circuitry 170. The transmission circuitry 110, the reception circuitry 120, the B-mode processing circuitry 130, the Doppler processing circuitry 140, the image generating circuitry 150, the memory 160, and the processing circuitry 170 are connected so as to be able to communicate with one another.

The transmission circuitry 110 includes a pulser circuit and the like. The pulser circuit is configured to repeatedly generate a rate pulse used for forming a transmission ultrasound wave at a predetermined rate frequency (a Pulse Repetition Frequency [PRF]) and to output the generated rate pulse to the ultrasound probe 101. Further, the pulser circuit is configured to apply the drive signal (a drive pulse) to the ultrasound probe 101 with timing based on the rate pulses. Further, under the control of the processing circuitry 170, the transmission circuitry 110 is configured to output an amplitude value of the drive signal output by the pulser circuit. Further, under the control of the processing circuitry 170, the transmission circuitry 110 is configured to transmit, to the ultrasound probe 101, a delay amount to be applied to the ultrasound waves transmitted from the ultrasound probe 101.

The reception circuitry 120 includes an Analog/Digital (A/D) converter and a reception beam former. For example, after receiving the reflected-wave signals from the ultrasound probe 101, the A/D converter converts the reflected-wave signals into digital data. Subsequently, the reception beam former generates reflected-wave data by performing a phased adding process on pieces of digital data from different channels. After that, the reception beam former transmits the generated reflected-wave data to the B-mode processing circuitry 130 and to the Doppler processing circuitry 140.

The B-mode processing circuitry 130 is configured to receive the reflected-wave data output from the reception circuitry 120 and to generate data (B-mode data) in which signal intensities are expressed by degrees of brightness, by performing a logarithmic amplification process, an envelope detection process, and/or the like on the received reflected-wave data.

The Doppler processing circuitry 140 is configured to receive the reflected-wave data output from the reception circuitry 120 and to generate data (Doppler data) obtained by extracting moving member information such as average velocity, dispersion, power, and the like with respect to multiple points, by performing a frequency analysis to obtain velocity information from the received reflected-wave data and extracting blood flows, tissues, contrast agent echo components based on the Doppler effect.

The image generating circuitry 150 is configured to generate the ultrasound image data from the data generated by the B-mode processing circuitry 130 and the Doppler processing circuitry 140. For example, the image generating circuitry 150 is configured to generate B-mode image data in which intensities of the reflected waves are expressed with degrees of brightness, from the B-mode data generated by the B-mode processing circuitry 130. Further, for example, the image generating circuitry 150 is configured to generate Doppler image data expressing the moving member information, from the Doppler data generated by the Doppler processing circuitry 140. The Doppler image data may be velocity image data, dispersion image data, power image data, or image data combining any of these types of image data.

In this situation, the image generating circuitry 150 may further perform various types of processes on the generated ultrasound image data. For example, the image generating circuitry 150 may generate display-purpose ultrasound image data by performing a coordinate transformation process compliant with the ultrasound scan mode used by the ultrasound probe 101. In one example, the image generating circuitry 150 generates the display-purpose ultrasound image data by converting (by performing a scan convert process) a scanning line signal sequence from an ultrasound scan into a scanning line signal sequence in a video format used by, for example, television. Further, for example, the image generating circuitry 150 performs an image processing process (a smoothing process) to re-generate a brightness average value image, an image processing process (an edge enhancement process) that uses a differential filter inside an image, or the like, by using a plurality of image frames resulting from the scan convert process. Also, for example, the image generating circuitry 150 combines text information of various parameters, scale graduations, body marks, and the like, with the ultrasound image data.

The memory 160 is realized by using, for example, a semiconductor memory element such as a Random Access Memory (RAM) or a flash memory, or a hard disk, an optical disk, or the like. For example, the memory 160 is configured to store therein the data (e.g., the B-mode image data and the Doppler image data) generated by the B-mode processing circuitry 130 and the Doppler processing circuitry 140, as well as the image data (e.g., the B-mode image data and the Doppler image data) generated by the image generating circuitry 150. Further, for example, the memory 160 is configured to store therein various types of data such as patient information, diagnosis protocols, various types of body marks, and the like. Further, for example, the memory 160 stores therein computer programs (hereinafter “programs”) used by various circuits included in the ultrasound diagnosis apparatus 10 to realize the functions thereof.

The memory 160 may be realized by one or more servers (a cloud) connected to the ultrasound diagnosis apparatus 10 via a network. Further, the one or more servers may be connected to a plurality of ultrasound diagnosis apparatuses so as to transmit and receive data to and from the plurality of ultrasound diagnosis apparatuses. In that situation, the one or more servers may directly be connected or may indirectly be connected to the plurality of ultrasound diagnosis apparatuses. For example, the one or more servers are able to transmit and receive the data to and from the plurality of ultrasound diagnosis apparatuses, via a management server that integrally manages the plurality of ultrasound diagnosis apparatuses provided in the hospital.

The processing circuitry 170 is configured to control operations of the entirety of the ultrasound diagnosis apparatus 10. For example, the processing circuitry 170 is configured to execute a controlling function 171, an obtaining function 172, and a display controlling function 173. The obtaining function 172 is an example of the obtaining unit. The display controlling function 173 is an example of the display controlling unit.

For example, the processing circuitry 170 is configured to control various functions of the processing circuitry 170 on the basis of an input operation received from the user via the input interface 102, by reading and executing a program corresponding to the controlling function 171 from the memory 160. For example, the processing circuitry 170 is configured to acquire the ultrasound image data, by controlling processes performed by the transmission circuitry 110, the reception circuitry 120, the B-mode processing circuitry 130, the Doppler processing circuitry 140, the image generating circuitry 150, and the like, on the basis of the various types of setting requests received from the user.

Further, for example, the processing circuitry 170 is configured to obtain information about a degree of deterioration of each ultrasound probe 101, by reading and executing a program corresponding to the obtaining function 172 from the memory 160. Further, for example, the processing circuitry 170 displays pieces of identification information (hereinafter, simply “IDs”) of a plurality of ultrasound probes 101, in a ranking order according to the information about the degrees of deterioration, by reading and executing a program corresponding to the display controlling function 173 from the memory 160. The obtainment of the information about the degree of deterioration and the display of the ranking order of the IDs will be explained later. Further, for example, the processing circuitry 170 causes the display 103 to display the display-purpose ultrasound image data generated by the image generating circuitry 150.

In the ultrasound diagnosis apparatus 10 illustrated in FIG. 2, the processing functions are stored in the memory 160 in the form of computer-executable programs. Each of the circuitry illustrated in FIG. 2 is a processor configured to realize the function thereof corresponding to one of the programs, by reading and executing the program from the memory 160. In other words, each of the circuitry that has read the corresponding program has the function corresponding to the read program.

With reference to FIG. 2, the example was explained in which the single processing circuit (i.e., the processing circuitry 170) realizes the processing functions represented by the controlling function 171, the obtaining function 172, and the display controlling function 173. However, possible embodiments are not limited to this example. For instance, another arrangement is also acceptable in which the processing circuitry 170 is structured by combining together a plurality of independent processors, so that the processing functions are realized as a result of the processors executing the programs. Further, the processing functions of the processing circuitry 170 may be realized as being distributed into a plurality of circuits or as being integrated into a single circuit, as appropriate.

The term “processor” used in the explanations above denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions thereof by reading and executing the programs saved in the memory 160.

The present disclosure has so far been explained based on the configuration in which the single memory (i.e., the memory 160) stores therein the programs corresponding to the functions of the circuitry illustrated in FIG. 2. However, possible embodiments are not limited to this example. For instance, more than one memory 160 may be arranged in a distributed manner. In that situation, each of the circuitry illustrated in FIG. 2 is configured to read a corresponding program from one of the individual memories 160 so as to realize the function thereof. Further, instead of saving the programs into the one or more memories 160, it is also acceptable to directly incorporate the programs in the circuitry of the one or more processors. In that situation, the one or more processors realize the functions by reading and executing the programs incorporated in the circuitry thereof.

Further, the circuitry illustrated in FIG. 2 may realize the functions thereof by using a processor of an external apparatus connected via a network. For example, the processing circuitry 170 realizes the functions, by reading and executing the programs corresponding to the functions illustrated in FIG. 2 from the memory 160 and further using an external workstation or a group of servers (a cloud) connected to the ultrasound diagnosis apparatus 10 via a network, as a computation resource.

An exemplary configuration of the ultrasound diagnosis system 1 has thus been explained. The ultrasound diagnosis apparatus 10 included in the ultrasound diagnosis system 1 configured as described above makes it easy to understand the degrees of deterioration of the ultrasound probes 101. More specifically, the ultrasound diagnosis apparatus 10 is configured to obtain the information about the degree of deterioration of each ultrasound probe 101 and to display IDs of the plurality of ultrasound probes 101 in a ranking order according to the obtained information. In the following sections, processes performed by the ultrasound diagnosis apparatus 10 according to the first embodiment will be explained in detail.

First, the obtaining function 172 obtains the information about the degree of deterioration of each ultrasound probe 101. In this situation, the information about the degree of deterioration is, for example, a period of time (hereinafter “operation time”; “periods of operation time” in the plural) during which the ultrasound probe 101 has been used. To be more specific, when an ultrasound probe 101 is used, the transducer elements vibrate to generate ultrasound waves. The transducer elements gradually become deteriorated due to the vibration. Accordingly, the longer the operation time of an ultrasound probe 101 is, the higher is the degree of deterioration of the ultrasound probe 101.

Other examples of the information about the degree of deterioration besides the operation time of the ultrasound probe 101 include an acoustic output of the ultrasound probe 101, an elapsed time period since the ultrasound probe 101 was manufactured, and frequency of use of the ultrasound probe 101. Further, when the ultrasound probe 101 includes a battery, a deterioration status of the battery can also serve as an example of the information about the degree of deterioration. Further, when the ultrasound probe 101 includes the input interface 102, a deterioration status of the input interface 102 can also serve as an example of the information about the degree of deterioration. The input interface 102 included in the ultrasound probe 101 is an example of the input unit. Examples of the input interface 102 in the ultrasound probe 101 include a freeze button provided on the ultrasound probe 101. In the present embodiment, the operation time of the ultrasound probes 101 will be explained, as an example of the information about the degrees of deterioration.

For example, the obtaining function 172 is configured to store, into the memory 160, operation time of each of the plurality of ultrasound probes 101. In one example, the obtaining function 172 first stores an ID of each of the plurality of ultrasound probes 101 into the memory 160. Each of the IDs of the ultrasound probes 101 serves as information used for identifying a different one of the plurality of ultrasound probes 101. Examples of the IDs include serial numbers each of which is appended to a different one of the plurality of ultrasound probes 101. Further, in correspondence with the ID, the obtaining function 172 arranges the operation time of each of the plurality of ultrasound probes 101 to be stored. After that, when one of the plurality of ultrasound probes 101 is used, the obtaining function 172 updates the operation time by adding the period of time during which the ultrasound probe 101 was newly used, to the operation time stored in the memory 160.

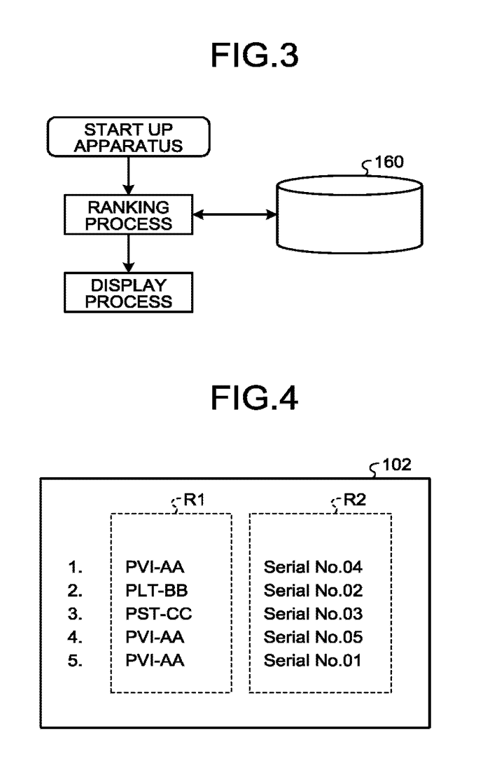

Next, an example of a process performed by the processing circuitry 170 after the ultrasound diagnosis apparatus 10 is started up will be explained with reference to FIG. 3. FIG. 3 is a drawing for explaining examples of a ranking process and a display process according to the first embodiment.

As illustrated in FIG. 3, after the ultrasound diagnosis apparatus 10 is started up, the obtaining function 172 obtains the operation time of each of the ultrasound probes 101 from the memory 160 and performs a ranking process. For example, the obtaining function 172 at first obtains the operation time of each of the plurality of ultrasound probes 101 in correspondence with the ID of the ultrasound probe 101. Subsequently, the obtaining function 172 specifies a ranking order of the IDs of the plurality of ultrasound probes 101, in descending order of the operation time.

In this situation, the obtaining function 172 may perform the ranking process on all the plurality of ultrasound probes 101 that can be connected to the apparatus main body 100 or may perform the ranking process on a part of the ultrasound probes 101. In one example, with respect to all the plurality of ultrasound probes 101 provided in the hospital or the like, the obtaining function 172 specifies the ranking order of the IDs of the plurality of ultrasound probes 101, in descending order of the operation time. In another example, with respect to certain ultrasound probes 101 among those provided in the hospital or the like excluding such ultrasound probes 101 that have not been used for a period of time, the obtaining function 172 specifies a ranking order of the IDs of the ultrasound probes 101, in descending order of the operation time.

After the ranking process is performed by the obtaining function 172, the display controlling function 173 performs a display process. In other words, the display controlling function 173 causes the display 103 to display the IDs of the plurality of ultrasound probes in the ranking order specified in the ranking process.

In this situation, the display controlling function 173 may perform the display process on all the plurality of ultrasound probes 101 that can be connected to the apparatus main body 100 or may perform the display process on a part of the ultrasound probes 101. In one example, when the ranking process was performed on certain ultrasound probes 101 excluding such ultrasound probes 101 that have not been used for a period of time, the display controlling function 173 displays, with respect to the certain ultrasound probes 101 subject to the ranking process, the IDs of the ultrasound probes 101 in a ranking order specified in the ranking process. In other words, the display controlling function 173 displays, with respect to the ultrasound probes 101 excluding the ultrasound probes 101 that have not been used for the certain period of time, the IDs of the ultrasound probes 101 in the ranking order specified in the ranking process.

In another example, when the ranking process was performed on all the plurality of ultrasound probes 101 that can be connected to the apparatus main body 100, the display controlling function 173 displays, with respect to all the plurality of ultrasound probes 101 subject to the ranking process, the IDs of the plurality of ultrasound probes in the ranking order specified in the ranking process. In yet another example, when the ranking process was performed on all the plurality of ultrasound probes 101 that can be connected to the apparatus main body 100, the display controlling function 173 may display, with respect to certain ultrasound probes 101 among the plurality of ultrasound probes 101 subject to the ranking process excluding such ultrasound probes 101 that have not been used for a period of time, the IDs of the ultrasound probes in a ranking order specified in the ranking process.

For example, as illustrated in FIG. 4, according to the ranking order specified in the ranking process, the display controlling function 173 displays the IDs of five ultrasound probes 101 having longer operation time among the plurality of ultrasound probes 101. FIG. 4 is a drawing illustrating the example of the displaying of the ranking order according to the first embodiment.

In a region R2 in FIG. 4, serial numbers of the five ultrasound probes 101 are indicated. The serial numbers in FIG. 4 serve as examples of the IDs of the ultrasound probes 101. In the following sections, the ultrasound probe 101 identified with “Serial No. 01” will be referred to as an ultrasound probe 1011. Similarly, the ultrasound probe 101 identified with “Serial No. 02” will be referred to as an ultrasound probe 1012. Also, the ultrasound probe 101 identified with “Serial No. 03” will be referred to as an ultrasound probe 1013. The ultrasound probe 101 identified with “Serial No. 04” will be referred to as an ultrasound probe 1014. The ultrasound probe 101 identified with “Serial No. 05” will be referred to as an ultrasound probe 1015. In other words, FIG. 4 indicates that the lengths of the operation time of the ultrasound probes become shorter in the following order: the ultrasound probe 1014 identified with “Serial No. 04”, the ultrasound probe 1012 identified with “Serial No. 02”, the ultrasound probe 1013 identified with “Serial No. 03”, the ultrasound probe 1015 identified with “Serial No. 05”, and the ultrasound probe 1011 identified with “Serial No. 01”.

Further, in a region R1 in FIG. 4, a probe ID of each of the five ultrasound probes 101 is indicated. In this situation, each of the probe IDs is information indicating the type of the ultrasound probe 101. In other words, the ultrasound probe 1014 identified with “Serial No. 04”, the ultrasound probe 1015 identified with “Serial No. 05”, and the ultrasound probe 1011 identified with “Serial No. 01” are the same types of ultrasound probes categorized as “PVI-AA”. In contrast, the ultrasound probe 1012 identified with “Serial No. 02” is an ultrasound probe categorized as “PLT-BB”. Further, the ultrasound probe 1013 identified with “Serial No. 03” is an ultrasound probe categorized as “PST-CC”. Alternatively, the display controlling function 173 may be configured not to display the region R1.

Among the plurality of ultrasound probes 101 provided in the hospital or the like, when there is no ultrasound probe 101 sharing a probe ID with another ultrasound probe 101, the obtaining function 172 and the display controlling function 173 may use the probe IDs as the identification information (the IDs). In other words, when there is only one ultrasound probe 101 corresponding to each probe ID, the obtaining function 172 may obtain the operation time of each of plurality of ultrasound probes 101 in correspondence with the probe ID thereof, so as to perform the ranking process. Further, the display controlling function 173 causes the display 103 to display the probe IDs of the plurality of ultrasound probes 101 in the ranking order specified in the ranking process. In that situation, the display controlling function 173 may be configured not to display the region R2. Alternatively, the display controlling function 173 may be configured to display, in the region R1, a product name of each of the ultrasound probes 101, in addition to or in place of the probe IDs.

For example, after the ultrasound diagnosis apparatus 10 is started up, the display controlling function 173 displays the ranking order illustrated in FIG. 4, before the user arranges a screen to be displayed to perform a log on operation. In another example, after the user is logged on, the display controlling function 173 may display the ranking order illustrated in FIG. 4, before the user arranges a screen to be displayed to input patient information.

As a result of the IDs of the plurality of ultrasound probes 101 being displayed in the ranking order, the user is able to easily understand the degree of deterioration of each of the plurality of ultrasound probes 101. For example, by referring to the display of the ranking order in FIG. 4, the user is able to easily understand that the operation time of the ultrasound probe 1014 identified with “Serial No. 04” is longer, and thus the degree of deterioration thereof is high. Further, when selecting an ultrasound probe 101 to be used in a medical examination, the user is able to avoid ultrasound probes 101 having higher degrees of deterioration. Accordingly, the ultrasound diagnosis apparatus 10 is able to avoid the situation where image quality of the ultrasound image data would be degraded by an ultrasound scan performed by using an ultrasound probe 101 having a high degree of deterioration and is thus able to improve efficiency of medical examinations.

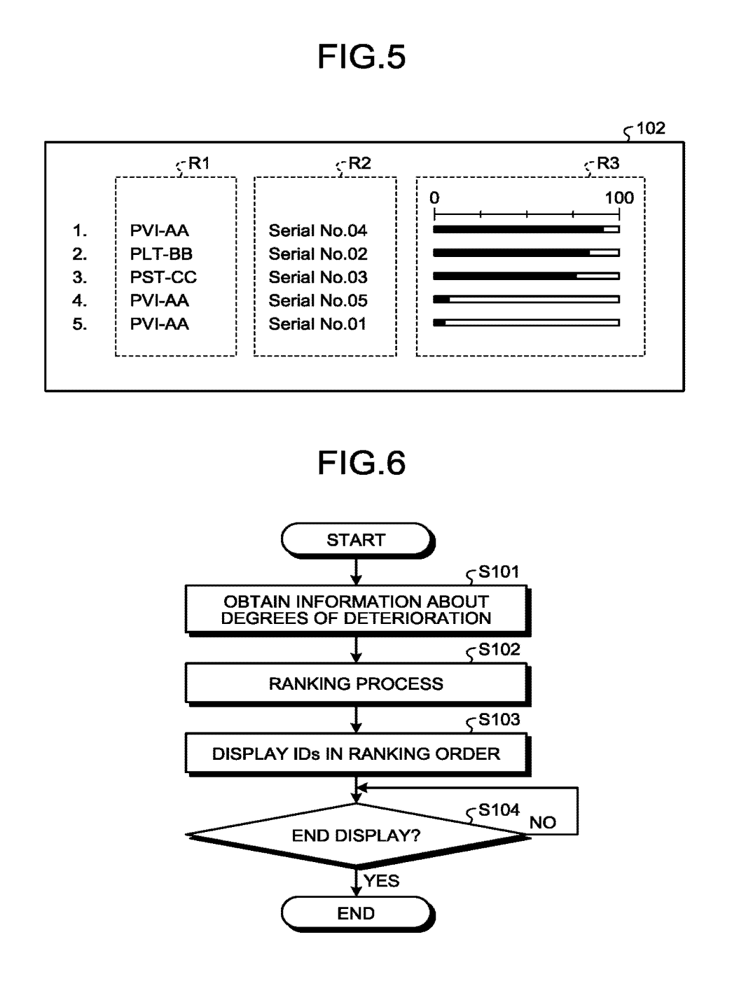

Further, the display controlling function 173 may be configured to display the information about the degree of deterioration of each of the ultrasound probes 101 in correspondence with the ID of the ultrasound probe 101. For example, as illustrated in FIG. 5, the display controlling function 173 may display the probe IDs in the region R1, display the serial numbers in the region R2, and also display meters indicating the degrees of usage of the ultrasound probes 101 in a region R3. FIG. 3 is a drawing illustrating the present example of the displaying of the ranking order according to the first embodiment.

More specifically, the obtaining function 172 at first obtains an operable time period for each of the probe IDs. In this situation, the operable time period denotes, for example, the length of time during which it is possible to acquire ultrasound image data having stable image quality by using one ultrasound probe 101. In one example, when ultrasound scans are performed by using a plurality of ultrasound probes 101 of mutually the same type, the operable time period denotes the operation time with which the percentage of ultrasound probes 101 causing image quality degradation in the ultrasound image data exceeds a threshold value. For example, the operable time period may be stored in a memory included in the ultrasound probe 101, in advance. In that situation, when the ultrasound probe 101 is connected to the apparatus main body 100, the obtaining function 172 obtains the operable time period from the memory included in the ultrasound probe 101 and stores the value into the memory 160.

Subsequently, the obtaining function 172 calculates, for each of the ultrasound probes 101, a ratio (a degree of usage) of the operation time to the operable time period. For example, the obtaining function 172 calculates the degree of usage in a numerical value range from “0” to “100”, by dividing the operation time by the operable time period and converting the result into a percentage value.

In one example, with respect to the ultrasound probe 1011 identified with “Serial No. 01”, the ultrasound probe 1014 identified with “Serial No. 04”, and the ultrasound probe 1015 identified with “Serial No. 05”, the obtaining function 172 calculates a degree of usage by dividing the operation time of each ultrasound probe by an operable time period of “PVI-AA” and converting the result into a percentage value. Further, the obtaining function 172 calculates a degree of usage by dividing the operation time of the ultrasound probe 1012 identified with “Serial No. 02” by an operable time period of “PLT-BB” and converting the result into a percentage value. Similarly, the obtaining function 172 calculates a degree usage by dividing the operation time of the ultrasound probe 1013 identified with “Serial No. 03” by an operable time period of “PST-CC” and converting the result into a percentage value.

Further, as illustrated in the region R3 of FIG. 5, the display controlling function 173 displays the meter indicating the degree of usage of each of the plurality of ultrasound probes 101. Alternatively, the display controlling function 173 may display numerals indicating the degrees of usage, in place of the meters in FIG. 5. Further, instead of the degrees of usage, the display controlling function 173 may display a meter indicating the operation time or a numeral indicating the operation time of each of the plurality of ultrasound probes 101.

The example was explained in which the ranking process is performed to arrange the periods of operation time in the descending order; however, possible embodiments are not limited to this example. For instance, the obtaining function 172 may specify a ranking order of the IDs of the plurality of ultrasound probes 101 in descending order of the degrees of usage. Alternatively, the obtaining function 172 may specify a ranking order of the IDs of the plurality of ultrasound probes 101 in ascending order of the operation time or the degrees of usage.

Further, the example was explained in which the operation time of each of the ultrasound probes 101 is obtained so as to calculate the degree of usage on the basis of the operation time; however, possible embodiments are not limited to this example. For instance, the obtaining function 172 may obtain the degree of usage of each of the ultrasound probes 101 from the memory 160.

In the following sections, a value (e.g., the degree of usage) calculated on the basis of the operation time of an ultrasound probe 101 and the operation time of the ultrasound probe 101 will collectively be referred to as an index value using the operation time. In other words, for each ultrasound probe 101, the obtaining function 172 is configured to obtain an index value using the operation time of the ultrasound probe 101. Further, the display controlling function 173 is configured to display the IDs of the plurality of ultrasound probes 101 in a ranking order according to the index values using the periods of operation time of the ultrasound probes 101. Further, the display controlling function 173 is configured to display the index value using the operation time for each of the IDs of the ultrasound probes 101.

As a result of the index values using the periods of operation time being displayed, the user is able to more specifically understand the degree of deterioration of each of the plurality of ultrasound probes 101. For example, by referring to the display of the ranking order illustrated in FIG. 5, the user is able to understand that the degrees of deterioration of the ultrasound probes 101 identified with “Serial No. 04”, “Serial No. 02”, and “Serial No. 03” are higher. In that situation, as an ultrasound probe 101 to be used in a medical examination, the user is able to select an ultrasound probe 101 other than those identified with “Serial No. 04”, “Serial No. 02”, and “Serial No. 03”. Consequently, the ultrasound diagnosis apparatus 10 is able to avoid the situation where image quality of the ultrasound image data would be degraded by an ultrasound scan performed by using an ultrasound probe 101 having a high degree of deterioration and is thus able to improve efficiency of medical examinations.

Further, because the degrees of usage of the ultrasound probes 101 identified with “Serial No. 04”, “Serial No. 02”, and “Serial No. 03” are close to “100”, the user is able to request a maintenance service or a component part replacement or to order a new ultrasound probe 101. Further, because the degrees of usage of the ultrasound probes 101 identified with “Serial No. 05” and “Serial No. 01” are low, the user is able to assess that a maintenance service and the like are not highly required.

Further, as illustrated in FIG. 5, among the ultrasound probes 101 identified with the probe ID “PVI-AA”, the degrees of usage of the ultrasound probe 1015 identified with “Serial No. 05” and the ultrasound probe 1011 identified with “Serial No. 01” are lower. Accordingly, the user is able to assess that, although the degree of usage of the ultrasound probe 1014 identified with “Serial No. 04” is higher, making a request for a component part replacement or ordering a new product is not highly required by the ultrasound probes 101 identified with the probe ID “PVI-AA”. In other words, the user is able to use the display in FIG. 5 as business management information of the hospital or the like.

As other examples, the index value using the operation time may be calculated by further using information other than the operation time. For example, the obtaining function 172 may at first obtain a parameter corresponding to an environment in which the ultrasound probe 101 is used. In one example, the obtaining function 172 obtains a parameter configured in such a manner that the larger the difference from an ordinary room temperature is, the larger is the value thereof. Further, the obtaining function 172 calculates a product of a parameter value corresponding to the room temperature in which an ultrasound probe 101 was used and the operation time, as an index value using the operation time.

Further, for example, the index value using the operation time may be calculated by further using various types of information such as an acoustic output of the ultrasound probe 101, an elapsed time period since the ultrasound probe 101 was manufactured, frequency of use of the ultrasound probe 101, a deterioration status of a battery, a deterioration status of the input interface 102, or the like. In other words, the obtaining function 172 is configured to obtain, for each ultrasound probe 101, an index value using at least the operation time.

Next, an example of a procedure in processes performed by the ultrasound diagnosis apparatus 10 will be explained with reference to FIG. 6. FIG. 6 is a flowchart for explaining a flow of a series of processes performed by the ultrasound diagnosis apparatus 10 according to the first embodiment. Steps S101 and S102 are steps corresponding to the obtaining function 172. Further, steps S103 and S104 are steps corresponding to the display controlling function 173.

First, after the ultrasound diagnosis apparatus 10 is started up, the processing circuitry 170 obtains information about the degree of deterioration of each ultrasound probe 101 (step S101). For example, for each ultrasound probe 101, the processing circuitry 170 obtains an index value using the operation time of the ultrasound probe 101, from the memory 160. Subsequently, the processing circuitry 170 performs the ranking process on the basis of the information about the degrees of deterioration (step S102).

After that, according to a result of the ranking process, the processing circuitry 170 displays a ranking order of the IDs of a plurality of ultrasound probes 101 (step S103). In this situation, the processing circuitry 170 may further display the information about the degree of deterioration, in correspondence with each of the IDs of the ultrasound probes 101. For example, for each of the IDs of the ultrasound probes 101, the processing circuitry 170 displays an index value (the operation time, the degree of usage, or the like) using the operation time of the ultrasound probe 101.

Subsequently, the processing circuitry 170 judges whether or not the display is to be ended (step S104). When having determined that the display is not to be ended (step S104: No), the processing circuitry 170 goes into a standby state. On the contrary, when having determined that the display is to be ended (step S104: Yes), the processing circuitry 170 ends the process. For example, when having received an input operation from the user indicating that the display is to be ended, the processing circuitry 170 ends the process.

As explained above, according to the first embodiment, the obtaining function 172 is configured to obtain the information about the degree of deterioration of each ultrasound probe 101. Further, the display controlling function 173 is configured to display the IDs of the plurality of ultrasound probes 101 in the ranking order according to the information about the degrees of deterioration. Accordingly, the ultrasound diagnosis apparatus 10 according to the first embodiment is able to make it easy to understand the degrees of deterioration of the ultrasound probes 101.

Further, the display controlling function 173 is further configured to display the information about the degree of deterioration in correspondence with each of the IDs of the ultrasound probes 101. Accordingly, the ultrasound diagnosis apparatus 10 according to the first embodiment makes it possible to understand the degrees of deterioration of the ultrasound probes 101 in an easy and specific manner.

In the first embodiment described above, the example is explained in which the ranking order is displayed regardless of purposes of use (hereinafter “purposes”) of the ultrasound probes 101. In contrast, in a second embodiment, an example will be explained in which a ranking order is displayed in accordance with purposes of the ultrasound probes 101.

The ultrasound diagnosis apparatus 10 according to the second embodiment has a configuration similar to that of the ultrasound diagnosis apparatus 10 illustrated in FIG. 2, except that a part of the processes performed by the obtaining function 172 and the display controlling function 173 is different. In the following sections, some of the constituent elements having the same configurations as those explained in the first embodiment will be referred to by using the same reference characters used in FIG. 1 or 2, and the explanations thereof will be omitted.

For example, prior to a medical examination to be performed on the patient P, the obtaining function 172 obtains patient information of the patient P. In one example, after the user is logged on, the display controlling function 173 causes the display 103 to display a screen used for inputting the patient information and further receives an input operation to input the patient information. As a result, the obtaining function 172 obtains the patient information of the patient P. In another example, after the user is logged on, the obtaining function 172 identifies examination targets of the user who was logged on, by connecting to a system such as the HIS. In this situation, when the examination targets of the user include the patient P, the obtaining function 172 obtains the patient information of the patient P from the system such as the HIS.

Subsequently, on the basis of the patient information, the obtaining function 172 specifies a purpose of the ultrasound probe 101 in the medical examination (hereinafter “predetermined purpose”). For example, on the basis of the patient information, the obtaining function 172 specifies the type of the ultrasound probe 101 (e.g., a linear probe, a convex probe, or a sector probe) to be used in the medical examination, as the predetermined purpose. Alternatively, for example, on the basis of the patient information, the obtaining function 172 specifies the examination target region (e.g., the thyroid gland, the abdomen, the heart) of the patient P in the medical examination, as the predetermined purpose.

After that, the obtaining function 172 specifies a plurality of ultrasound probes 101 corresponding to the predetermined purpose. For example, when a type of ultrasound probes 101 is specified as the predetermined purpose, the obtaining function 172 specifies a plurality of ultrasound probes 101 corresponding to the specified type. For example, when the type of the ultrasound probe 101 to be used in the medical examination is a “linear probe”, the obtaining function 172 specifies a plurality of ultrasound probes 101 that are each a “linear probe” from among the ultrasound probes 101 that can be connected to the apparatus main body 100.

Further, when the examination target region is specified as the predetermined purpose, the obtaining function 172 specifies a plurality of ultrasound probes 101 that can be used for examining the specified examination target region. For example, when the examination target region is “the thyroid gland”, the obtaining function 172 specifies a plurality of ultrasound probes 101 that can be used for examining “the thyroid glad” from among the ultrasound probes 101 that can be connected to the apparatus main body 100. In one example, to examine “the thyroid gland”, a “linear probe” is commonly used. Accordingly, the obtaining function 172 specifies a plurality of ultrasound probes 101 that are each a “linear probe”.

In the following sections, an example will be explained in which ultrasound probes 101 identified with the probe ID “PLT-BB” are each a “linear probe”. For example, as a plurality of ultrasound probes 101 that are each a “linear probe”, the obtaining function 172 specifies four ultrasound probes 101 identified with the probe ID “PLT-BB” (namely, the ultrasound probe 1012 identified with “Serial No. 02”, an ultrasound probe 1016 identified with “Serial No. 06”, an ultrasound probe 1017 identified with “Serial No. 07”, and an ultrasound probe 1018 identified with “Serial No. 08”).

Subsequently, the obtaining function 172 performs a ranking process on the specified plurality of ultrasound probes 101, on the basis of the information about the degree of deterioration. For example, with respect to the specified plurality of ultrasound probes 101, the obtaining function 172 specifies a ranking order of the IDs of the plurality of ultrasound probes 101, in descending order of the operation time.

After the ranking process is performed by the obtaining function 172, the display controlling function 173 performs the display process. In other words, the display controlling function 173 causes the display 103 to display the IDs of the plurality of ultrasound probes 101 specified by the obtaining function 172, in the ranking order according to the information about the degrees of deterioration.

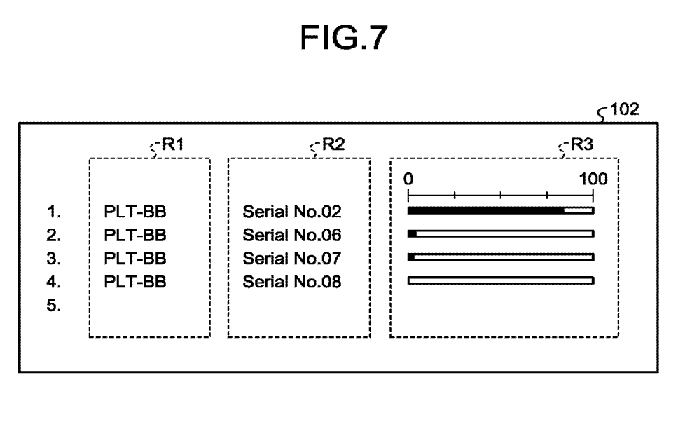

For example, as illustrated in FIG. 7, the display controlling function 173 displays the IDs of four ultrasound probes 101 specified by the obtaining function 172, according to a ranking order specified by the ranking process. FIG. 7 is a drawing illustrating the example of the displaying of the ranking order according to the second embodiment.

For example, in the region R1 in FIG. 7, the display controlling function 173 displays the probe ID of each of the four ultrasound probes 101. As illustrated in FIG. 7, the four ultrasound probes 101 are ultrasound probes of mutually the same type categorized as “PLT-BB”. In other words, the four ultrasound probes 101 are each a “linear probe”.

Further, in the region R2 in FIG. 7, the display controlling function 173 displays a serial number of each of the four ultrasound probes 101. The serial numbers illustrated in FIG. 7 serve as examples of IDs of the ultrasound probes 101. For example, FIG. 7 indicates that the lengths of the operation time become shorter in the following order: the ultrasound probe 1012 identified with “Serial No. 02”, the ultrasound probe 1016 identified with “Serial No. 06”, the ultrasound probe 1017 identified with “Serial No. 07”, and the ultrasound probe 1018 identified with “Serial No. 08”.

Further, in the region R3 in FIG. 7, the display controlling function 173 displays a meter indicating the degree of usage of each of the four ultrasound probes 101. Alternatively, in place of the meters illustrated in FIG. 7, the display controlling function 173 may display numerals indicating the degrees of usage. Further, in place of the degree of usage, the display controlling function 173 may display a meter indicating the operation time or a numeral indicating the operation time of each of the plurality of ultrasound probes 101.

As a result of the IDs of the plurality of ultrasound probes 101 corresponding to the predetermined purpose being displayed in the ranking order, the user is able to understand the degrees of deterioration more easily, with respect to the plurality of ultrasound probes 101 corresponding to the predetermined purpose. For example, when a “linear probe” is to be used in a medical examination to be performed, the user selects one of the plurality of “linear probes”. In response to this, the ultrasound diagnosis apparatus 10 displays a ranking order with resect to the plurality of “linear probes”. In other words, the ultrasound diagnosis apparatus 10 is capable of displaying the ranking order of only the “linear probes” in which the user is interested, while omitting the display of other ultrasound probes 101 (e.g., “convex probes” and “sector probes”) not corresponding to the predetermined purpose.

Further, in a large-scale hospital or the like, the number of ultrasound probes 101 that can be connected to the apparatus main body 100 may be larger than the number of ultrasound probes 101 that can be displayed on the display 103 at the same time. In that situation, the display controlling function 173 arranges the display 103 to display only a part of the ultrasound probes 101 that can be connected to the apparatus main body 100. For example, as illustrated in FIG. 4, among the plurality of ultrasound probes 101, the display controlling function 173 displays, in a ranking order, the IDs of only the five ultrasound probes 101 having longer operation time.

In this situation, by displaying, in the ranking order, the IDs of the plurality of ultrasound probes 101 corresponding to the predetermined purpose, the display controlling function 173 is able to display, with a higher priority, the IDs of the ultrasound probes 101 in which the user is interested. For example, as illustrated in FIG. 7, the display controlling function 173 is also able to display the IDs of the ultrasound probe 1016 identified with “Serial No. 06”, the ultrasound probe 1017 identified with “Serial No. 07”, and the ultrasound probe 1018 identified with “Serial No. 08”, which were not displayed in the example in FIG. 4. As a result, for example, the user is able to understand that, although the degree of deterioration of the ultrasound probe 1012 identified with “Serial No. 02” is high, it is possible to use the ultrasound probe 1016 identified with “Serial No. 06”, the ultrasound probe 1017 identified with “Serial No. 07”, and the ultrasound probe 1018 identified with “Serial No. 08” each having a low degree of deterioration.

Further, as illustrated in FIG. 7, among the plurality of ultrasound probes 101 that are each a “linear probe”, the degrees of usage of the ultrasound probe 1016 identified with “Serial No. 06” and the ultrasound probe 1017 identified with “Serial No. 07” are low. Further, the ultrasound probe 1018 identified with “Serial No. 08” has hardly been used. Accordingly, the user is able to assess that, although the degree of usage of the ultrasound probe 1012 identified with “Serial No. 02” is high, making a request for a component part replacement or ordering a new product is not highly required for the “linear probes”. Further, because the “linear probes” include the ultrasound probe 1018 that has hardly been used, the user is able to make a judgement that the number of products to be ordered in the future shall be decreased, or the like. In other words, the user is able to use the display in FIG. 7 as business management information of the hospital or the like.

The example was explained above in which the predetermined purpose is specified on the basis of the patient information; however, possible embodiments are not limited to this example. For instance, the obtaining function 172 may receive an input operation to input the predetermined purpose from the user.

Next, another example of displaying a ranking order according to purposes will be explained. For example, the obtaining function 172 first categorizes the plurality of ultrasound probes 101 that can be connected to the apparatus main body 100 into groups in correspondence with purposes thereof. In one example, the obtaining function 172 categorizes the plurality of ultrasound probes 101 into the groups in correspondence with the types thereof. In another example, the obtaining function 172 categorizes the plurality of ultrasound probes 101 into the groups in correspondence with examination target regions for which certain types of ultrasound probes 101 are commonly used. In this situation, the obtaining function 172 may categorize one ultrasound probe 101 into a plurality of groups.

Subsequently, the obtaining function 172 performs a ranking process for each of the purposes. For example, the obtaining function 172 performs a ranking process on a plurality of ultrasound probes 101 categorized as “linear probes”. Further, the obtaining function 172 performs another ranking process on a plurality of ultrasound probes 101 categorized as “convex probes”. Furthermore, the obtaining function 172 performs yet another ranking process on a plurality of ultrasound probes 101 categorized as “sector probes”.

After that, the display controlling function 173 displays the IDs of the plurality of ultrasound probes 101 in the ranking order for each of the purposes. For example, the display controlling function 173 displays a ranking order for the plurality of ultrasound probes 101 categorized as the “linear probes”, for the plurality of ultrasound probes 101 categorized as the “convex probes”, and for the plurality of ultrasound probes 101 categorized as the “sector probes”.

As a result of the IDs of the plurality of ultrasound probes 101 being displayed in the ranking order for each of the purposes, the user is able to more easily understand the degrees of deterioration, with respect to the plurality of ultrasound probes 101 corresponding to the purpose in which the user is interested. Further, when the IDs of the plurality of ultrasound probes 101 are displayed in the ranking order for each of the purposes, there is no need to specify a predetermined purpose. In other words, even before the user is logged on when an input of the patient information or a predetermined purpose has not yet been received from the user, the ultrasound diagnosis apparatus 10 is able to display the IDs of the plurality of ultrasound probes 101 in the ranking order for each of the purposes.

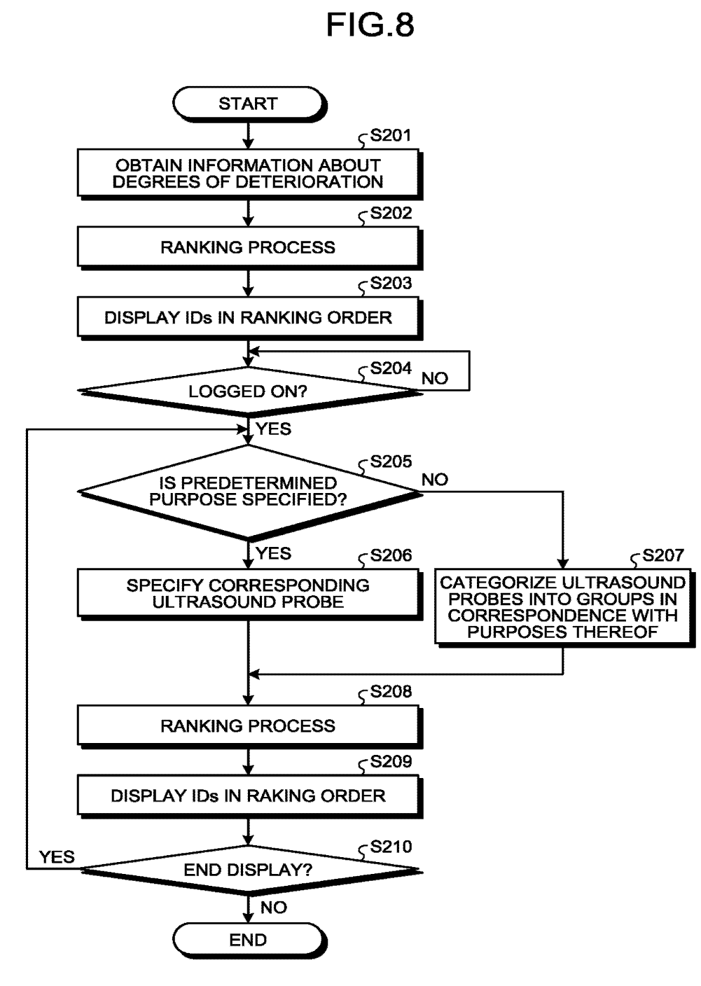

Next, an example of a procedure in processes performed by the ultrasound diagnosis apparatus 10 will be explained with reference to FIG. 8. FIG. 8 is a flowchart for explaining a flow of a series of processes performed by the ultrasound diagnosis apparatus 10 according to the second embodiment. Steps S201, S202, S204, S205, S206, S207, and S208 are steps corresponding to the obtaining function 172. Further, steps S203, S209, and S210 are steps corresponding to the display controlling function 173.

At first, after the ultrasound diagnosis apparatus 10 is started up, the processing circuitry 170 at first obtains information about the degree of deterioration of each ultrasound probe 101 (step S201) and performs the ranking process on the basis of the information about the degrees of deterioration (step S202). Subsequently, according to a result of the ranking process, the processing circuitry 170 displays the IDs of a plurality of ultrasound probes 101 in a ranking order (step S203).

In this situation, the processing circuitry 170 judges whether or not the user is logged on (step S204). When the user is not logged on (step S204: No), the processing circuitry 170 goes into a standby state. On the contrary, when the user is logged on (step S204: Yes), the processing circuitry 170 judges whether or not a predetermined purpose is specified (step S205). For example, when an input of the patient information is received from the user or when the patient information is obtained from a system such as the HIS, the processing circuitry 170 specifies the predetermined purpose on the basis of the patient information and determines that the predetermined purpose is specified. Also, when an input of a predetermined purpose is received from the user, the processing circuitry 170 determines that the predetermined purpose is specified.

When the predetermined purpose is specified (step S205: Yes), the processing circuitry 170 specifies a plurality of ultrasound probes 101 corresponding to the predetermined purpose (step S206). On the contrary, when the predetermined purpose is not specified (step S205: No), the processing circuitry 170 categorizes the plurality of ultrasound probes 101 into groups in correspondence with the purposes thereof (step S207).

After the process at step S206 or S207 is performed, the processing circuitry 170 performs the ranking process (step S208). For example, when it was determined at step S205 that the predetermined purpose is specified, the processing circuitry 170 performs the ranking process on a plurality of ultrasound probes 101 corresponding to the predetermined purpose. On the contrary, when it was determined at step S205 that the predetermined purpose is not specified, the processing circuitry 170 performs the ranking process for each of the purposes.

Subsequently, the processing circuitry 170 displays the IDs of the plurality of ultrasound probes 101 in a ranking order (step S209). For example, when it was determined at step S205 that the predetermined purpose is specified, the processing circuitry 170 displays the IDs of the plurality of ultrasound probes 101 corresponding to the predetermined purpose in a ranking order. On the contrary, when it was determined at step S205 that the predetermined purpose is not specified, the processing circuitry 170 displays the IDs of the plurality of ultrasound probes 101 in a ranking order for each of the purposes.

Subsequently, the processing circuitry 170 judges whether or not the display is to be ended (step S210). When it is determined that the display is not to be ended (step S210: No), the processing circuitry 170 returns to step S205. On the contrary, when it is determined that the display is to be ended (step S210: Yes), the processing circuitry 170 ends the process.

Alternatively, the processing circuitry 170 may display the ranking order in correspondence with each of the purposes of the ultrasound probes 101 at step S203. For example, before step S202, the processing circuitry 170 categorizes the plurality of ultrasound probes 101 into groups in correspondence with the purposes thereof. After that, the processing circuitry 170 performs a ranking process for each of the purposes at step S202. Subsequently, at step S203, the processing circuitry 170 displays the IDs of a plurality of ultrasound probes 101 in a ranking order for each of the purposes.

In yet another example, before step S202, the processing circuitry 170 may receive an input of the patient information from the user and specify a predetermined purpose on the basis of the patient information. Alternatively, before step S202, the processing circuitry 170 may receive an input of a predetermined purpose from the user. Subsequently, at step S202, the processing circuitry 170 performs a ranking process on the plurality of ultrasound probes 101 corresponding to the predetermined purpose. After that, at step S203, the processing circuitry 170 displays the IDs of the plurality of ultrasound probes 101 corresponding to the predetermined purpose, in a ranking order.

As explained above, according to the second embodiment, the obtaining function 172 is configured to specify the plurality of ultrasound probes 101 corresponding to the predetermined purpose. Further, the display controlling function 173 is configured to display the IDs of the specified plurality of ultrasound probes 101 in the ranking order according to the information about the degrees of deterioration. Consequently, the ultrasound diagnosis apparatus 10 according to the second embodiment is able to make it easier to understand the degrees of deterioration, with respect to the ultrasound probes 101 in which the user is interested.

Alternatively, the obtaining function 172 is configured to categorize the plurality of ultrasound probes 101 into the groups in correspondence with the purposes thereof. Further, the display controlling function 173 is configured to display the IDs of the plurality of ultrasound probes 101 in correspondence with each of the purposes, in the ranking order according to the information about the degrees of deterioration. Consequently, the ultrasound diagnosis apparatus 10 according to the second embodiment is able to make it easier to understand the degrees of deterioration, with respect to the ultrasound probes 101 in which the user is interested, without using the predetermined purpose.

In the first and the second embodiments above, the example is explained in which the information about the degree of deterioration of each of the ultrasound probes 101 is obtained, regardless of which user used each of the ultrasound probes 101. In contrast, in a third embodiment, an example will be explained in which the information about the degree of deterioration of each of the ultrasound probes 101 is obtained in correspondence with users.

The ultrasound diagnosis apparatus 10 according to the third embodiment has a configuration similar to that of the ultrasound diagnosis apparatus 10 illustrated in FIG. 2, except that a part of the processes performed by the obtaining function 172 and the display controlling function 173 is different. In the following sections, some of the constituent elements having the same configurations as those explained in the first embodiment will be referred to by using the same reference characters used in FIG. 1 or 2, and the explanations thereof will be omitted.

For example, the obtaining function 172 stores, into the memory 160, the operation time of each of the plurality of ultrasound probes 101 in correspondence with users. In one example, the obtaining function 172 at first stores, into the memory 160, sets each made up of the ID of a different one of the plurality of ultrasound probes 101 and a user.

In the following sections, an example will be explained in which there are five ultrasound probes 101 that can be connected to the apparatus main body 100, namely, the ultrasound probe 1011 identified with “Serial No. 01”, the ultrasound probe 1012 identified with “Serial No. 02”, the ultrasound probe 1013 identified with “Serial No. 03”, the ultrasound probe 1014 identified with “Serial No. 04”, and the ultrasound probe 1015 identified with “Serial No. 05”. Further, in the present example, the users who use these five ultrasound probes 101 are three medical doctors, namely, Doctor A, Doctor B, and Doctor C.

In this situation, the obtaining function 172 stores, into the memory 160, a set made up of “Serial No. 01” and Doctor A, a set made up of “Serial No. 02” and Doctor A, a set made up of “Serial No. 03” and Doctor A, a set made up of “Serial No. 04” and Doctor A, and a set made up of “Serial No. 05” and Doctor A. Further, the obtaining function 172 stores, into the memory 160, a set made up of “Serial No. 01” and Doctor B, a set made up of “Serial No. 02” and Doctor B, a set made up of “Serial No. 03” and Doctor B, a set made up of “Serial No. 04” and Doctor B, and a set made up of “Serial No. 05” and Doctor B. In addition, the obtaining function 172 stores, into the memory 160, a set made up of “Serial No. 01” and Doctor C, a set made up of “Serial No. 02” and Doctor C, a set made up of “Serial No. 03” and Doctor C, a set made up of “Serial No. 04” and Doctor C, and a set made up of “Serial No. 05” and Doctor C.

Further, the obtaining function 172 arranges the periods of operation time of the ultrasound probes 101 to be stored in correspondence with these sets. For example, the obtaining function 172 arranges the time period during which Doctor A has used the ultrasound probe 1011 identified with “Serial No. 01” to be stored in correspondence with the set made up of “Serial No. 01” and Doctor A. After that, when Doctor A used the ultrasound probe 1011, the obtaining function 172 updates the operation time by adding the time period during which the ultrasound probe 1011 was newly used, to the operation time stored in the memory 160.

For example, after the ultrasound diagnosis apparatus 10 is started up, the obtaining function 172 obtains, from the memory 160, the operation time for each of the sets made up of the ID of an ultrasound probe 101 and a user and further performs ranking processes. In one example, the obtaining function 172 performs a ranking process on the operation time of the ultrasound probe 1011 used by Doctor A, the operation time of the ultrasound probe 1012 used by Doctor A, the operation time of the ultrasound probe 1013 used by Doctor A, the operation time of the ultrasound probe 1014 used by Doctor A, and the operation time of the ultrasound probe 1015 used by Doctor A. For example, the obtaining function 172 specifies a ranking order of the IDs of the five ultrasound probes 101 to be in descending order of the lengths of the operation time by Doctor A.

Similarly, the obtaining function 172 performs a ranking process on the operation time of the ultrasound probe 1011 used by Doctor B, the operation time of the ultrasound probe 1012 used by Doctor B, the operation time of the ultrasound probe 1013 used by Doctor B, the operation time of the ultrasound probe 1014 used by Doctor B, and the operation time of the ultrasound probe 1015 used by Doctor B. Also, similarly, the obtaining function 172 performs a ranking process on the operation time of the ultrasound probe 1011 used by Doctor C, the operation time of the ultrasound probe 1012 used by Doctor C, the operation time of the ultrasound probe 1013 used by Doctor C, the operation time of the ultrasound probe 1014 used by Doctor C, and the operation time of the ultrasound probe 1015 used by Doctor C.

Further, the obtaining function 172 performs a ranking process on total operation time of the ultrasound probe 1011, total operation time of the ultrasound probe 1012, total operation time of the ultrasound probe 1013, total operation time of the ultrasound probe 1014, and total operation time of the ultrasound probe 1015. In other words, the obtaining function 172 performs the ranking process on the periods of operation time of the ultrasound probes 101, regardless of which user used the ultrasound probes 101.

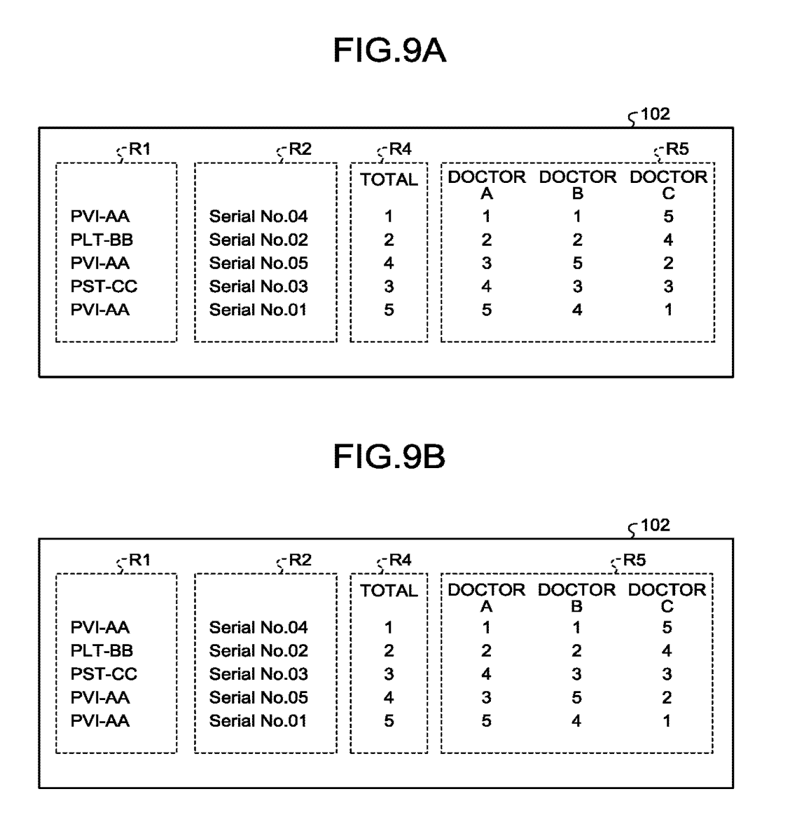

Subsequently, the display controlling function 173 displays the IDs of the plurality of ultrasound probes 101 in the ranking order according to the operation time of each of the users. For example, as illustrated in FIG. 9A, the display controlling function 173 displays the IDs of the plurality of ultrasound probes 101 in the ranking order according to the operation time by Doctor A. FIG. 9A is a drawing illustrating the present example of the displaying of the ranking order according to the third embodiment. In FIG. 9A, the region R1 indicates the probe ID of each of the five ultrasound probes 101. Further, the region R2 in FIG. 9A indicates the serial number of each of the five ultrasound probes 101.

A region R4 in FIG. 9A indicates the ranking order of the total operation time by the plurality of users. In other words, the region R4 in FIG. 9A indicates that the lengths of the total operation time become shorter in the following order: the ultrasound probe 1014 identified with “Serial No. 04”, the ultrasound probe 1012 identified with “Serial No. 02”, the ultrasound probe 1013 identified with “Serial No. 03”, the ultrasound probe 1015 identified with “Serial No. 05”, and the ultrasound probe 1011 identified with “Serial No. 01”. In this situation, the ranking order of the total operation time indicated in the region R4 is a value calculated on the basis of the operation time of the ultrasound probes 101 and therefore serves as an example of the index value using the operation time. In other words, the ranking order of the total operation time is an example of the information about the degree of deterioration of each of the ultrasound probes 101.

Further, a region R5 in FIG. 9A indicates a ranking order of the periods of operation time of each of the users. More specifically, the region R5 in FIG. 9A indicates that the lengths of the operation time by Doctor A become shorter in the following order: the ultrasound probe 1014 identified with “Serial No. 04”, the ultrasound probe 1012 identified with “Serial No. 02”, the ultrasound probe 1015 identified with “Serial No. 05”, the ultrasound probe 1013 identified with “Serial No. 03”, and the ultrasound probe 1011 identified with “Serial No. 01”.

Also, the region R5 in FIG. 9A indicates that the lengths of the operation time by Doctor B become shorter in the following order: the ultrasound probe 1014 identified with “Serial No. 04”, the ultrasound probe 1012 identified with “Serial No. 02”, the ultrasound probe 1013 identified with “Serial No. 03”, the ultrasound probe 1011 identified with “Serial No. 01”, and the ultrasound probe 1015 identified with “Serial No. 05”.

In addition, the region R5 in FIG. 9A indicates that the lengths of the operation time by Doctor C become shorter in the following order: the ultrasound probe 1011 identified with “Serial No. 01”, the ultrasound probe 1015 identified with “Serial No. 05”, the ultrasound probe 1013 identified with “Serial No. 03”, the ultrasound probe 1012 identified with “Serial No. 02”, and the ultrasound probe 1014 identified with “Serial No. 04”.

In this situation, the ranking order of the periods of operation time of each of the users indicated in the region R5 of FIG. 9A is a value calculated on the basis of the operation time of the ultrasound probes 101 and therefore serves as an example of the index value using the operation time. In other words, the ranking order of the periods of operation time of each of the users is an example of the information about the degree of deterioration of each of the ultrasound probes 101.

As illustrated in FIG. 9A, as a result of the IDs of the plurality of ultrasound probes 101 being displayed in the ranking order according to the operation time by Doctor A, Doctor A is able to easily understand the ultrasound probe 101 that he/she has used more. Further, Doctor A is able to easily understand the total operation time of each of the ultrasound probes 101 that he/she has used. For example, as indicated in FIG. 9A, the ultrasound probe 1014 identified with “Serial No. 04” that has been used by Doctor A for the longest period of time also has long total operation time. Accordingly, Doctor A is able to understand that the ultrasound probe 101 that he/she has used more has a high degree of deterioration and to consider changing which ultrasound probe 101 is to be used, or the like.

Further, in FIG. 9A, the ultrasound probes 101 that have been used for longer periods of time by Doctor A and Doctor B also have a tendency to have longer total operation time. In contrast, the ultrasound probes 101 that have been used for longer periods of time by Doctor C have a tendency to have shorter total operation time. In other words, compared to Doctors A and B, it is safe to say that Doctor C is properly selecting such ultrasound probes 101 that have lower degrees of deterioration. Accordingly, by referring to the information about Doctor C, Doctors A and B are able to consider changing which ultrasound probe 101 is to be used, or the like.

With reference to FIG. 9A, the example was explained in which the IDs of the plurality of ultrasound probes 101 are displayed in the ranking order according to the operation time by Doctor A. However, possible embodiments are not limited to this example. For instance, the display controlling function 173 may display the IDs of the plurality of ultrasound probes 101 in the ranking order according to the operation time by Doctor B or in the ranking order according to the operation time by Doctor C.

Further, with reference to FIG. 9A, the ranking order of the periods of operation time for each of the users is displayed for the three medical doctors, namely Doctor A, Doctor B, and Doctor C; however, possible embodiments are not limited to this example. For instance, the display controlling function 173 may display the ranking order of the periods of operation time of each of the users only for two medical doctors among Doctors A, B, and C or only for one medical doctor. Further, the display controlling function 173 may omit the display in the region R4.

Further, with reference to FIG. 9A, the example was explained in which the IDs of the plurality of ultrasound probes 101 are displayed in the ranking order according to the operation time of each of the users; however, possible embodiments are not limited to this example. For instance, as indicated in FIG. 9B, the display controlling function 173 may display the IDs of the plurality of ultrasound probes 101 in a ranking order according to the total operation time. FIG. 9B is a drawing illustrating the present example of the displaying of the ranking order according to the third embodiment.

Further, with reference to FIGS. 9A and 9B, the example was explained in which the operation time is obtained for each of the sets made up of the ID of an ultrasound probe 101 and a user; however, possible embodiments are not limited to this example. For instance, the obtaining function 172 may obtain, for each of the sets made up of the ID of an ultrasound probe 101 and a user, the degree of usage of the ultrasound probe 101. Further, for example, the obtaining function 172 may obtain a ranking order of the periods of operation time or a ranking order of the degrees of usage of the ultrasound probes 101, for the sets each made up of the ID of an ultrasound probe 101 and a user.

Further, with reference to FIGS. 9A and 9B, the example was explained in which the ranking orders of the periods of operation time are displayed in the regions R4 and R5; however, possible embodiments are not limited to this example. For instance, the display controlling function 173 may display the periods of operation time or the degrees of usage in the regions R4 and R5.

The first to the third embodiments have thus been explained. It is, however, also possible to carry out the present disclosure in various different modes other than those described in the first to the third embodiments.

In the embodiments described above, the serial numbers and the probe IDs are explained as examples of the identification information (the IDs). However, possible embodiments are not limited to this example. For instance, the IDs may each be a name or the like that is appended by the user to each of the plurality of ultrasound probes 101. Further, for example, each of the IDs may be an image of the corresponding one of the plurality of ultrasound probes 101.

Further, in the embodiments described above, the user is assumed to be a medical doctor or a medical technologist; however, possible embodiments are not limited to this example. For instance, the user may be a service person who provides a maintenance service for the ultrasound probes 101. In that situation, the ultrasound diagnosis apparatus 10 displays the IDs of the ultrasound probes 101 in a ranking order for the service person. Accordingly, the service person is able to easily understand the degrees of deterioration of the ultrasound probes 101 and to replace component parts of ultrasound probes 101 having higher degrees of deterioration and to optimize the cycle with which the ultrasound probes 101 receive a maintenance service.

Further, the display controlling function 173 may change the displayed information in accordance with users. For example, when the user is a medical doctor or a medical technologist, the display controlling function 173 displays only minimum information. In one example, when the user is a medical doctor or a medical technologist, the display controlling function 173 displays only the IDs of the plurality of ultrasound probes 101 in a ranking order. In contrast, when the user is a service person, the display controlling function 173 displays the IDs of the plurality of ultrasound probes 101 in a ranking order, and also displays various types of information about the degree of deterioration of the ultrasound probes 101. With these arrangements, the service person is able to more specifically understand the state of use or the like of the ultrasound probes 101 at the time of a breakdown of an ultrasound probe 101, or the like.

Further, in the embodiments described above, the index values using the operation time of the ultrasound probes 101 are explained, as the information about the degree of deterioration of each of the ultrasound probes 101. However, possible embodiments are not limited to this example.

For instance, as the information about the degree of deterioration of each of the ultrasound probes 101, the obtaining function 172 may obtain, for each ultrasound probe 101, an index value using an acoustic output of the ultrasound probe 101. To be more specific, even when the periods of operation time are the same, the larger the energy of an output ultrasound wave is, the higher is the degree of deterioration of the ultrasound probe 101. Accordingly, as the information about the degree of deterioration of each of the ultrasound probes 101, the obtaining function 172 obtains the index value using the acoustic output of the ultrasound probe 101.

For example, when the acoustic output is too large during a harmonic imaging process using a contrast agent (microbubbles or bubbles), the contrast agent may burst. Accordingly, during such a harmonic imaging process using a contrast agent, the ultrasound probe 101 outputs an ultrasound wave having a smaller level of energy than that used in an ultrasound scan or the like performed for acquiring B-mode image data. For example, during such a harmonic imaging process using a contrast agent, the drive voltage of the ultrasound probe 101 is approximately 10 V, and the degree of deterioration caused per unit time period is small.

Further, for example, during a Shear Wave Elastography (SWE) process performed to measure and render in an image the level of firmness of a tissue in a patient's body, a displacement is caused by a shear wave by applying an acoustic radiation force or mechanical vibration to the patient's tissue from the body surface, so as to measure the propagation speed and an elastic modulus of the shear wave, by chronologically measuring the displacements in various points on a scanned cross-sectional plane. In that situation, the ultrasound probe 101 outputs a burst wave (a push pulse) to cause the displacements with the shear wave. When the push pulse is output, the drive voltage of the ultrasound probe 101 may be 100 V or higher in some situations, and the degree of deterioration per unit time period is large.

In one example, as the information about the degree of deterioration of each of the ultrasound probes 101, the obtaining function 172 calculates a product of the operation time and the acoustic output of the ultrasound probe 101. In other words, as the information about the degree of deterioration of each of the ultrasound probes 101, the obtaining function 172 is configured to calculate the index value using at least the operation time of the ultrasound probe 101 and the acoustic output of the ultrasound probe 101. Alternatively, as the information about the degree of deterioration of each of the ultrasound probes 101, the obtaining function 172 may obtain, from the memory 160, the index value using at least the operation time of the ultrasound probe 101 and the acoustic output of the ultrasound probe 101.

Further, for example, as the information about the degree of deterioration of each of the ultrasound probes 101, the obtaining function 172 may obtain, for each ultrasound probe 101, an index value using an elapsed time period since the ultrasound probe 101 was manufactured. To be more specific, even when the periods of operation time are the same, the longer the elapsed time period since the manufacture is, the higher is the degree of deterioration of the ultrasound probe 101. Accordingly, as the information about the degree of deterioration of each of the ultrasound probes 101, the obtaining function 172 calculates an index value using at least the elapsed time period since the manufacture of the ultrasound probe 101. Alternatively, as the information about the degree of deterioration of each of the ultrasound probes 101, the obtaining function 172 may obtain, from the memory 160, an index value using at least the elapsed time period since the manufacture of the ultrasound probe 101.

Further, for example, as the information about the degree of deterioration of each of the ultrasound probes 101, the obtaining function 172 may obtain, for each ultrasound probe 101, an index value using a deterioration status of a battery included in the ultrasound probe 101. To be more specific, wireless ultrasound probes 101 perform ultrasound scans with energy supplied from a battery installed therein. In this situation, when the battery becomes deteriorated, the time period during which the ultrasound probe 101 remains operable without being electrically charged becomes shorter, and it becomes difficult to use the ultrasound probe 101. Accordingly, as the information about the degree of deterioration of each of the ultrasound probes 101, the obtaining function 172 obtains the index value using the deterioration status of the battery included in the ultrasound probe 101.

In one example, the obtaining function 172 obtains the capacity of the battery, as the deterioration status of the battery. For example, the obtaining function 172 obtains the capacity of the battery, by estimating the capacity of the battery on the basis of the number of times the battery has been charged and discharged. In another example, the obtaining function 172 obtains the capacity of the battery by measuring the amount of electric power that can be discharged after one session of charging. Further, as the information about the degree of deterioration of each of the ultrasound probes 101, the obtaining function 172 calculates an index value using at least the capacity of the battery. Alternatively, as the information about the degree of deterioration of each of the ultrasound probes 101, the obtaining function 172 may obtain, from the memory 160, the index value using at least the capacity of the battery.

Further, for example, as the information about the degree of deterioration of each of the ultrasound probes 101, the obtaining function 172 may obtain, for each ultrasound probe 101, an index value using a deterioration status of the input interface 102 included in the ultrasound probe 101. To be more specific, when an ultrasound probe 101 includes the input interface 102, if the input interface 102 becomes deteriorated, it becomes difficult to perform input operations, and it also becomes difficult to use the ultrasound probe 101 itself. Accordingly, as the information about the degree of deterioration of each of the ultrasound probes 101, the obtaining function 172 obtains the index value using the deterioration status of the input interface 102 included in the ultrasound probe 101.

In one example, as the deterioration status of the input interface 102, the obtaining function 172 obtains the number of times a freeze button has been used. Further, the obtaining function 172 calculates, as the information about the degree of deterioration of each of the ultrasound probes 101, an index value using at least the number of times the freeze button has been used. Alternatively, as the information about the degree of deterioration of each of the ultrasound probes 101, the obtaining function 172 may obtain, from the memory 160, the index value using at least the number of times the freeze button has been used.

Further, for example, as the information about the degree of deterioration of each of the ultrasound probes 101, the obtaining function 172 may obtain, for each ultrasound probe 101, an index value using frequency of use (e.g., operation time per unit time period) of the ultrasound probe 101. To be more specific, even when accumulated periods of operation time (hereinafter, “accumulative operation time”) are the same, degrees of deterioration of the ultrasound probes 101 vary depending on the frequency of use. Accordingly, as the information about the degree of deterioration of each of the ultrasound probes 101, the obtaining function 172 obtains an index value using at least the frequency of use of the ultrasound probe 101. Alternatively, as the information about the degree of deterioration of each of the ultrasound probes 101, the obtaining function 172 may obtain, from the memory 160, the index value using at least the frequency of use of the ultrasound probe 101.

It is possible to use any of the various types of index values described above in arbitrary combination. Further, the obtaining function 172 may obtain the various types of index values described above individually or as a single index value. For example, the obtaining function 172 individually obtains two or more of the various types of index values described above and further generates a single index value based on the obtained two or more types of index values. In one example, the obtaining function 172 may generate the single index value by totaling the obtained two or more types of index values. In this situation, the obtaining function 172 may generate the single index value by applying weights to the obtained two or more types of index values and further totaling the weighted index values. In other words, as the information about the degrees of deterioration of the ultrasound probes 101, the obtaining function 172 calculates the value obtained by applying the predetermined weights to the index values and totaling the results. Alternatively, as the information about the degrees of deterioration of the ultrasound probes 101, the obtaining function 172 may obtain, from the memory 160, a weighted sum of the various types of index values described above. When applying the weights, the value of at least a part of the two or more types of index values may be fixed. In other words, the weight may be “1”.

Further, in the embodiments described above, the example is explained in which the memory 160 stores therein the information about the degree of deterioration of each of the ultrasound probes 101. However, possible embodiments are not limited to this example. For instance, a storage device (e.g., the image storing apparatus 20 or the server 30) connected to the ultrasound diagnosis apparatus 10 via a network may store therein the information about the degree of deterioration of each of the ultrasound probes 101.

Further, when a storage device is connected to a plurality of ultrasound diagnosis apparatuses 10, the storage device may be configured to store therein the information about the degree of deterioration of each ultrasound probe 101, with respect to a plurality of ultrasound probes 101 that are used by at least one of the plurality of ultrasound diagnosis apparatuses 10. Further, the obtaining function 172 obtains the information about the degree of deterioration of each of the ultrasound probes 101 from the storage device via a network.

For example, the storage device may be connected to a first ultrasound diagnosis apparatus and a second ultrasound diagnosis apparatus. Further, as the information about the degree of deterioration of each of the ultrasound probes 101, the storage device stores therein the operation time of the ultrasound probe 101.

In that situation, for example, the first ultrasound diagnosis apparatus accesses the storage device with arbitrary timing and obtains the operation time of each of the ultrasound probes 101. In other words, the first ultrasound diagnosis apparatus obtains the operation time of each of the ultrasound probes 101 by synchronizing with the storage device. The timing of the synchronization with the storage device may be when the first ultrasound diagnosis apparatus is started up (when being turned on) or shut down or at another predetermined time (e.g., when the hospital's business hours start or end). Similarly, the second ultrasound diagnosis apparatus is also able to obtain the operation time of each of the ultrasound probes 101. Further, when any of the plurality of ultrasound probes 101 is used, the storage device updates the operation time being stored.

For example, when synchronizing with the storage device, the first ultrasound diagnosis apparatus transmits, to the storage device, a time period during which an ultrasound probe 101 was newly used by the first ultrasound diagnosis apparatus. In this situation, the “time period during which an ultrasound probes 101 was newly used” denotes, for example, the time period during which the ultrasound probe 101 was used after the most recent synchronization with the storage device.

In one example, the first ultrasound diagnosis apparatus transmits, to the storage device, the time period during which the ultrasound probe 101 was newly used and the ID of the ultrasound probe 101 that was used. Further, on the basis of the information transmitted thereto from the first ultrasound diagnosis apparatus, the storage device updates the operation time in a database. In one example, the storage device first specifies the newly-used ultrasound probe 101 on the basis of the ID transmitted thereto from the first ultrasound diagnosis apparatus. Subsequently, the storage device updates the operation time in the database by adding the time period during which the ultrasound probe 101 was newly used, to the operation time in the database. In this situation, after synchronizing with the storage device, the ultrasound diagnosis apparatus resets the record of the time period during which the ultrasound probe 101 was newly used. Further, by synchronizing with the storage device, other apparatuses such as the second ultrasound diagnosis apparatus are able to obtain the operation time of the ultrasound probes 101 and to display a ranking order.

In this situation, the first ultrasound diagnosis apparatus may further transmit various types of information to the storage device. For example, the first ultrasound diagnosis apparatus transmits, to the storage device, information indicating the time at which the most recent synchronization was performed, together with the time period during which the ultrasound probe 101 was newly used and the ID of the ultrasound probe 101 that was used. With this arrangement, the storage device is able to avoid updating the operation time in duplicate, by judging whether or not the database already reflects the information transmitted thereto from the first ultrasound diagnosis apparatus. For example, the storage device manages a history of synchronization with the first ultrasound diagnosis apparatus in the database. Further, the storage device updates the operation time stored in the database, when the information indicating the time of the most recent synchronization transmitted thereto from the first ultrasound diagnosis apparatus matches the history of synchronization stored in the database.

Further, for example, when a sensitivity measuring process is performed on an ultrasound probe 101, the first ultrasound diagnosis apparatus may transmit sensitivity measurement data to the storage device. In one example, the first ultrasound diagnosis apparatus generates the sensitivity measurement data by taking an image of a biological phantom by using the ultrasound probe 101 and evaluating the obtained data. After that, when synchronizing with the storage device, the first ultrasound diagnosis apparatus transmits the sensitivity measurement data to the storage device. Further, the storage device registers the sensitivity measurement data transmitted thereto from the first ultrasound diagnosis apparatus into the database or updates sensitivity measurement data that is already registered. Further, by synchronizing with the storage device, other apparatuses such as the second ultrasound diagnosis apparatus are able to obtain the sensitivity measurement data of the ultrasound probe 101 and to present users with the data. By using the sensitivity measurement data, the users are able to more specifically understand the degree of deterioration of the ultrasound probe 101. For example, by referring to the sensitivity measurement data of a part or all of the ultrasound probes 101 displayed in a ranking order, the users are able to quantitatively understand the degree of deterioration of the ultrasound probes 101.

Further, in place of the time period during which the ultrasound probe 101 was newly used, the first ultrasound diagnosis apparatus may transmit accumulative operation time of the ultrasound probe 101 to the storage device. Further, on the basis of the operation time transmitted thereto from the first ultrasound diagnosis apparatus, the storage device updates the operation time stored in the database. When ultrasound probes 101 are used by a plurality of ultrasound diagnosis apparatuses before the synchronization with the storage device, there may be some situations where it is not possible to properly manage the accumulative operation time of the ultrasound probes 101. It is therefore desirable for the first ultrasound diagnosis apparatus to synchronize with the storage device every time an ultrasound probe 101 is newly used, so as to update the operation time stored in the storage device in a timely manner.

Further, the storage device may manage various types of data in addition to the accumulative operation time of the ultrasound probes 101. For example, the storage device may manage a history of synchronization in correspondence with IDs of ultrasound probes 101. In this situation, the history of synchronization indicates, for example, identification information of the synchronized apparatuses (e.g., the first ultrasound diagnosis apparatus and the second ultrasound diagnosis apparatus) and the times and dates of synchronization. Further, the storage device may manage difference values between before the synchronization and after the synchronization, in addition to the accumulative operation time. In other words, the storage device may be configured to manage the “time period during which the ultrasound probe 101 was newly used”.

The example is explained above in which the ultrasound diagnosis system 1 includes the two ultrasound diagnosis apparatuses; however, the number of ultrasound diagnosis apparatuses included in the ultrasound diagnosis system 1 may be three or more or may be one. Further, although the operation time was used as an example, the present disclosure is similarly applicable to other types of information related to a degree of deterioration of each of the ultrasound probes 101.

Further, for example, a memory included in the ultrasound probe 101 may be configured to store therein the information about the degree of deterioration of the ultrasound probe 101. For example, a memory included in the ultrasound probe 1011 stores therein the information about the degree of deterioration of the ultrasound probe 1011. Subsequently, when the ultrasound probe 1011 is connected to the apparatus main body 100, the obtaining function 172 stores the information about the degree of deterioration of the ultrasound probe 1011 into the memory 160. After that, when the ultrasound probe 1012 is connected to the apparatus main body 100, the obtaining function 172 stores the information about the degree of deterioration of the ultrasound probe 1011 into a memory included in the ultrasound probe 1012. With these arrangements, when the ultrasound probe 1012 is connected to the apparatus main body 100 again, the obtaining function 172 is able to obtain the information about the degree of deterioration of both the ultrasound probe 1011 and the ultrasound probe 1012.

The constituent elements of the apparatuses and the devices in the above embodiments are based on functional concepts. Thus, it is not necessary to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program that is analyzed and executed by the CPU or may be realized as hardware using wired logic.

Further, the controlling method explained in the above embodiments may be realized by causing a computer such as a personal computer or a workstation to execute a display controlling program prepared in advance. The display controlling program may be distributed via a network such as the Internet. Further, the display controlling program may be recorded on a non-transient computer-readable storage medium such as a hard disk, a flexible disk (FD), a Compact Disk Read-Only Memory (CD-ROM), a Magneto-Optical (MO) disk, or a Digital Versatile Disk (DVD), so as to be executed as being read from the storage medium by a computer.

According to at least one aspect of the embodiments described above, it is possible to make it easy to understand the degrees of deterioration of the ultrasound probes.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising processing circuitry configured:
to obtain information about a degree of deterioration of each ultrasound probe; and
to display pieces of identification information of a plurality of ultrasound probes in a ranking order according to the information.

2. The ultrasound diagnosis apparatus according to claim 1, wherein the processing circuitry further displays the information in correspondence with each of the pieces of identification information of the ultrasound probes.

3. The ultrasound diagnosis apparatus according to claim 1, wherein
the processing circuitry specifies two or more of the ultrasound probes corresponding to a predetermined purpose, and
the processing circuitry displays pieces of identification information of the specified plurality of ultrasound probes in a ranking order according to the information.

4. The ultrasound diagnosis apparatus according to claim 1, wherein
the processing circuitry categorizes the plurality of ultrasound probes into groups in correspondence with purposes thereof, and
the processing circuitry displays the pieces of identification information of the plurality of ultrasound probes in correspondence with each of the purposes, in the ranking order according to the information.

5. The ultrasound diagnosis apparatus according to claim 3, wherein the predetermined purpose is one selected from between an examination target region or a type of the ultrasound probe.

6. The ultrasound diagnosis apparatus according to claim 1, wherein
the processing circuitry obtains the information for each of users of the ultrasound probes,
the processing circuitry displays the pieces of identification information of the plurality of ultrasound probes in a ranking order according to a total value of the information of each of the users, and
the processing circuitry displays the information of each of the users for each of the pieces of identification information of the ultrasound probes.

7. The ultrasound diagnosis apparatus according to claim 1, wherein the processing circuitry obtains the information from a storage device configured to store therein, for each ultrasound probe, the information about the plurality of ultrasound probes being used by a plurality of ultrasound diagnosis apparatuses.

8. The ultrasound diagnosis apparatus according to claim 1, wherein
the processing circuitry obtains a plurality of types of index values individually as the information, and
the processing circuitry generates a single index value based on the plurality of types of index values.

9. The ultrasound diagnosis apparatus according to claim 8, wherein the processing circuitry generates the single index value by totaling the plurality of types of index values.

10. The ultrasound diagnosis apparatus according to claim 9, wherein the processing circuitry generates the single index value by applying weights to the plurality of types of index values and subsequently totaling the weighted plurality of types of index values.

11. The ultrasound diagnosis apparatus according to claim 1, wherein, as the information, the processing circuitry obtains, for each ultrasound probe, an index value using at least a period of operation time of the ultrasound probe.

12. The ultrasound diagnosis apparatus according to claim 11, wherein, as the information, the processing circuitry obtains, for each ultrasound probe, an index value using at least an acoustic output of the ultrasound probe.

13. The ultrasound diagnosis apparatus according to claim 11, wherein, as the information, the processing circuitry obtains, for each ultrasound probe, an index value using at least an elapsed time period since manufacture of the ultrasound probe.

14. The ultrasound diagnosis apparatus according to claim 1, wherein, as the information, the processing circuitry obtains, for each ultrasound probe, an index value using at least a deterioration status of a battery included in the ultrasound probe.

15. The ultrasound diagnosis apparatus according to claim 1, wherein, as the information, the processing circuitry obtains, for each ultrasound probe, an index value using at least a deterioration status of an input interface included in the ultrasound probe.

16. The ultrasound diagnosis apparatus according to claim 1, wherein, as the information, the processing circuitry obtains, for each ultrasound probe, an index value using at least frequency of use of the ultrasound probe.

17. The ultrasound diagnosis apparatus according to claim 1, wherein
the processing circuitry obtains the information for each of users of the ultrasound probes,
the processing circuitry displays the pieces of identification information of the plurality of ultrasound probes in a ranking order corresponding to the information of each of the users, and
the processing circuitry displays the information of each of the users for each of the pieces of identification information of the ultrasound probes.

18. The ultrasound diagnosis apparatus according to claim 1, wherein the processing circuitry obtains the information about each ultrasound probe from a memory included in the ultrasound probe.

19. A computer-readable storage medium recording thereon a display controlling program that causes a computer to perform processes of:
obtaining information about a degree of deterioration of each ultrasound probe; and
displaying pieces of identification information of a plurality of ultrasound probes in a ranking order according to the information.
